(19) United States
(12) Patent Application Publication
(10) Pub. No.: US 2008/0160543 A1
(43) Pub. Date: Jul. 3, 2008

(54) Electrochemical immunostrip and its preparation method

(76) Inventors: Chein-Shyong Su, Taipei (TW); Wen-Jing Cheng, Taipei (TW); Maio-Ling Hung, Taipei (TW); Tai-Guang Wu, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/645,556

(22) Filed: Dec. 27, 2006

(51) Int. Cl. G01N 33/53

(52) U.S. Cl. 435/7.9

(57) ABSTRACT

An electrochemical immuno diagnostic strip and its preparation method are disclosed herein. A screen-printed electrode is prepared on an insulating substrate, which is divided into an electrode area and a connecting area. An electrochemical precursor is placed on the electrode area and a reaction membrane is provided on the electrode area. A specimen loading area is at the reaction membrane. The present invention is advantageous to overcome the interference problem of the electrochemical detection technology and to provide the quantitative analysis of the prompt diagnostic strip.

### BACKGROUND OF THE INVENTION

1. Field of the Invention

The present invention generally relates to the biological detection method, and more particularly relates to an electrochemical strip for immuno diagnostic and its preparation method.

2. Description of the Prior Art

Electrochemical sensors, while used for other bioanalytical applications, have received only limited attention for nucleic acid analysis. However, electrochemical detection systems, if demonstrated to be accurate and reproducible, offer potential advantages for detection of DNA and RNA and for “lab-on-a-chip” devices, including high sensitivity and selectivity, ultra-small dead volumes, fast response, compatibility with advanced microfabrication and miniaturization technologies, low-cost, and minimal power requirements.

The U.S. Pat. No. 5,516,644 discloses a binding assay process in an electrochemical detector, called binding assay device, in which a substance to be assayed in a liquid test sample is determined by developing the substance in a liquid test sample in a matrix, which allows the substance to be assayed to react with a specific binding substance which has specific affinity for the substance to be assayed; and having a signal substance generator to chance its distribution in the matrix in response to the reaction of the substance with the binding one, then detecting the resulting distributional changes of signals which are rate-limited by the mass transfer of a signal substance generated from the signal substance generator.

The U.S. Pat. No. 5,516,644 discloses an electrochemical membrane strip biosensor, which combining the immuno-chromatographic method and the electric conductivity detection technology so that on-site quantitative determination at the points of care or at home may be carried out. But certain physical symptoms, such as pregnancy and ovulation, or bacterial infection may be identified by a qualitative analysis of indicating substances.

Accordingly to the continuously development of the sensing technology, the sensitivity, the convenience, and the cost for the sensing technology are important issues for the user. The various requirements of the medical equipment used in the laboratory center or hospital drive the design of the equipment to become huge and expensive. On the other hand, the diagnostic requirement is extended to the market of the home-use and on-site use. However, its development is limited by the present equipment design and the sensing technology. The current technology used in the automatical diagnostic equipment has seldom successful case to apply to the portable diagnostic equipment.

Currently, most of the immuno diagnostic strips are used in the qualitative analysis. For example, the pregnancy test strip only provides the result of yes or no and can not be memorized for the test result. The colorimetric assay is developed to overcome this problem, however, owing to the cutoff value limitation of the qualitative analysis of the original test strip, so that the sensitivity of the colorimetric assay is poor.

The electrochemical sensing technology is very simple and easy and the equipment design is suitable for home-use, the prompt small equipment for on-site detection or the application of the biosensor. However, the cross reaction is one of issues on the application of the electrochemical sensing technology, especially on the diagnosis the body fluid in which having other similar compounds, lives, chemical substances. It is difficult for electrochemical sensing to avoid the cross reaction of the biochemical compound within the specimen under the detection potential. For example, the amount of the uric acid or Vitamin C in the body fluid is quite high and it will cause the serious interferences.

Hence, the present invention is to provide an electrochemical immuno diagnostic strip and its preparation method, and then some disadvantages of the interference can be overcome by the immunoreaction.

### SUMMARY OF THE INVENTION

One of objects of the present invention is to provide an electrochemical immuno diagnostic strip and the preparation method thereof. Both the interference issue of the electrochemical detection is effectively overcome and the detection sensitivity is enhanced by utilizing the immunoreaction.

Another, one of objects of the present invention is to provide an electrochemical immuno diagnostic strip and its preparation method to provide a one-step and prompt detection strip and the quantitative analysis.

Further, one of objects of the present invention is to provide an electrochemical immuno diagnostic strip and its preparation method to combine the biotechnology with the electric manufacturing technology to provide a biosensors strip with low-cost, simple, easy, and convenience.

The present invention proposes an electrochemical immuno diagnostic strip including: an insulating substrate; a screen-printed electrode arranged on the insulating substrate, wherein the screen-printed electrode comprises an electrode area and a connecting area; an electrochemical precursor modified on the electrode area; and a reaction membrane. Wherein, a second end of the reaction membrane is stacked on the electrode area of the screen-printed electrode; an analyte capturing compound is fixed at the reaction membrane; a specimen loading area is at a first end of the reaction membrane for loading a specimen having an analyte therein, wherein an analyte/enzyme labeled compound is fixed on the specimen area; and the analyte/enzyme labeled compound is re-dissolved by the specimen and proceeds toward the direction of the second end of the reaction membrane; both of the analyte and the analyte/enzyme labeled compound are performing an immunoreaction with the analyte capturing compound; and the residual analyte/enzyme labeled compound is transported to the surface of the electrode area for catalyzing the electrochemical precursor to produce a electrochemical active material.

Another, the present invention proposes an electrochemical immuno diagnostic strip comprising: an insulating substrate; a screen-printed electrode arranged on the insulating substrate, wherein the screen-printed electrode comprises an electrode area and a connecting area; an electrochemical precursor modified on the electrode area; and a reaction membrane. Wherein, a second end of the reaction membrane is stacked on the electrode area on the screen-printed electrode; a complex of an analyte capturing compound and an analyte/enzyme labeled compound is fixed at the reaction membrane by the side of the analyte capturing compound of the complex; a specimen loading area arranged at a first end of the reaction membrane for loading a specimen having an analyte therein, wherein the specimen is proceeds toward the direction of the second end of the reaction membrane; the analyte substitutes the analyte/enzyme labeled compound from the complex formed by the analyte/enzyme labeled compound and the analyte capturing compound; and the replaced analyte/enzyme labeled compound is transported toward the surface of the electrode area to catalyze the electrochemical precursor producing an electrochemical active material.

Further, the present invention proposes a preparation method of an electrochemical immuno diagnostic strip comprising the following steps: providing an insulating substrate; forming a screen-printed electrode on the insulating substrate, wherein the screen-printed electrode comprises an electrode area and a connecting area; modifying an electrochemical precursor on the electrode area; and providing a reaction membrane, wherein a second end of the reaction membrane is stacked on the electrode area on the screen-printed electrode and a specimen loading area is arranged at a first end of the reaction membrane.

Other advantages of the present invention will become apparent from the following description taken in conjunction with the accompanying drawings wherein are set forth, by way of illustration and example, certain embodiments of the present invention.

### BRIEF DESCRIPTION OF THE DRAWINGS

The foregoing aspects and many of the accompanying advantages of this invention will become more readily appreciated as the same becomes better understood by reference to the following detailed description, when taken in conjunction with the accompanying drawings, wherein:

FIG. 1A is a schematic representation of the cross-section view illustrating the architecture of an electrochemical immuno diagnostic strip in accordance with one embodiment of the present invention;

FIG. 1B is a partial enlarge schematic representation of the FIG. 1A, in accordance with one embodiment of the present invention;

FIG. 2 is a schematic representation of the cross-section view illustrating the architecture of an electrochemical immuno diagnostic strip, in accordance with the other embodiment of the present invention;

FIG. 3 and FIG. 4 are schematic representations of the cross-section view illustrating the architecture of an electrochemical immuno diagnostic strip in accordance with another embodiment of the present invention;

FIG. 5 is a schematic representation of the cross-section view illustrating the architecture of an electrochemical immuno diagnostic strip in accordance with the further embodiment of the present invention; and

FIG. 6 is a schematic representation of the cross-section view of the architecture of an electrochemical immuno diagnostic strip in accordance with the furthermore embodiment of the present invention.

### DESCRIPTION OF THE PREFERRED EMBODIMENT

The present invention provides an electrochemical immuno diagnostic strip and its preparation method. A prompt and quantitative detection strip is provided with the resolution to effectively overcome the interference problem of the electrochemical analysis.

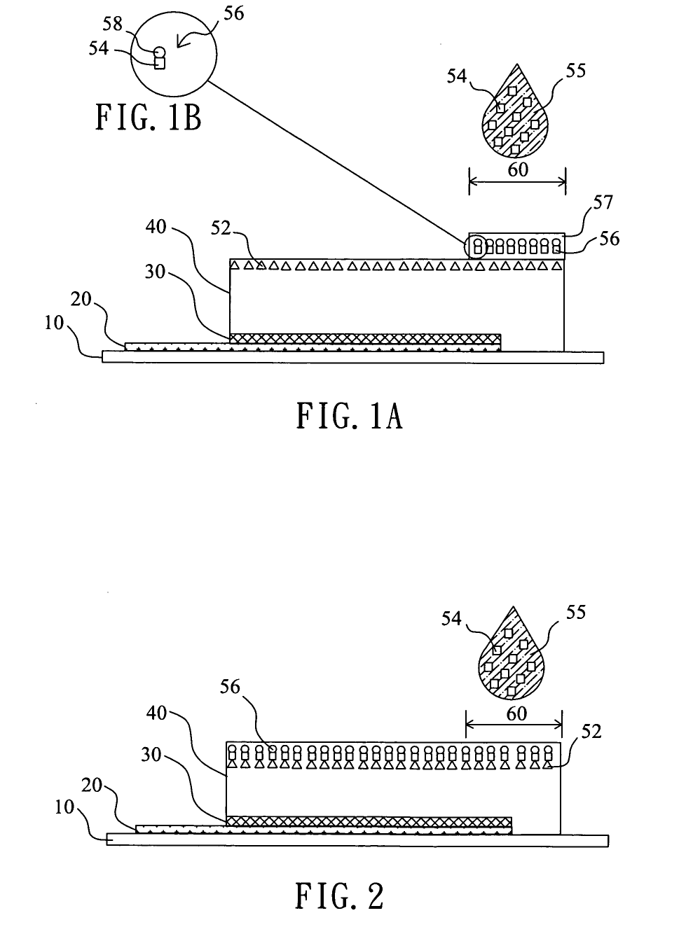

Referring to FIG. 1A, a screen-printed electrode 20 thereon is positioned on an insulating substrate 10. The screen-printed electrode 20 includes both an electrode area for modifying an electrochemical precursor 30 and a connecting area. One portion of a reaction membrane 40 is stacked on the electrode area of the screen-printed electrode 20. In one embodiment, the reaction membrane 40 is made of filter paper, non-woven, fiber film, or polymer material. Furthermore, an analyte capturing compound 52 is fixed atop the surface of the reaction membrane 40 on which a specimen loading area 60 is configured. A film 57 including an analyte/enzyme labeled compound 56 is arranged within the specimen loading area 60 of the reaction membrane 40.

Referring to FIG. 1B, the analyte/enzyme labeled compound 56 is a conjugate of the analyte 54 and a catalyst 58 for the electrochemical precursor 30. In one embodiment, the electrochemical precursor 30 may be the para-aminophenyl β-D-galactopyranoside (PAPG) and the catalyst 58 is the β-galactosidase. On the other hand, the specimen 55 carrying the analyte 54 may be loaded into the reaction membrane 40 through the specimen loading area 60.

Referring to FIG. 2, the difference from the embodiment of FIG. 1A is the analyte capturing compound 52 and an analyte/enzyme labeled compound 56 may be embedded in the reaction membrane 40 and bonding with each other.

In one embodiment of the present invention, the analyte capturing compound is an antibody on the condition that the analyte is an antigen. Alternatively, as the analyte is an antibody, the analyte capturing compound is an antigen. In the embodiment, the analyte may be the protein, such as the antibody, cancer factor or the hormone of the large molecule or the chemical medicine, the prohibit medicine, the antibiotics, such as the Flunitrazepam (FM2) or the Sulfamethazine (SMT), of the small molecule. Alternatively, the analyte capturing compound is an avidin on the condition that the analyte is a biotin (Vitamins H). The combination of the antibody and the antigen has a high specificity so as to effectively prevent the cross reaction of the analyte-similar compound in the specimen. Besides, the affinity of the biotin and the avidin is much higher than the specificity of the binding of antibody with antigen.

The SMT has the functions of the antiseptic, the anthelmintic, and the growth enhancement for the animal. However, it may cause serious effects on the human health if the people ingest the meat containing the residual SMT. The present diagnostic strip may be used for sifting the SMT amount from the serum. For example, the Flunitrazepam, so-called FM2 and composed of the benzodiazepine type medicine is one kind of the antidepressant drug, which causes a user in the addiction by the amount of overdose. Recently, evildoers further use the FM2 as the tool for raping because sufferers taking the drink with FM2 will lose their clear mind, sleep in comatose, or the amnesia, hence, the FM2 is more and more exactingly controlled. Next, Vitamin H is a water-soluble vitamin and involved in many carboxylase reactions in the humane body. As the amount of the Vitamin is too low in the blood or in the urine, it may indicate a rare disorder of the multiple carboxylase deficiency.

Accordingly, as the analyte is the FM2 (or the SMT), the analyte capturing compound is the combination of the antibody and the FM2 (or the SMT), and the analyte/enzyme labeled compound is the conjugate of the β-galactosidase and the FM2 (or the SMT). After the immunoreaction, the residual and free analyte/β-galactosidase labeled compound may catalyze and modify the electrochemical precursor on the surface of the electrode area with which an electrochemical active material is formed for the following electrochemical detection.

For example, as the analyte is the biotin (Vitamin H), the analyte capturing compound is the avidin, and the analyte/enzyme labeled compound is the conjugate of the β-galactosidase and the biotin. After the immuno reaction, the residual and free biotin/β-galactosidase labeled compound may catalyze and modify the electrochemical precursor (PAPG) on the electrode area with which an electrochemical active material is formed for the following electrochemical detection. Alternatively, the analyte capturing compound is the mouse-anti-human-IgG-antibody on the condition that the analyte is the human-IgG. Furthermore, the analyte/enzyme labeled compound is the conjugate of the β-galactosidase and the human-IgG. Similarly, after the immunoreaction, the residual and free human-IgG/β-galactosidase labeled compound may catalyze and modify the electrochemical precursor (PAPG) to produce an electrochemical active material for the following electrochemical detection.

The drawings of the present invention illustrate the embodiments with the elements in various sizes for clarity, not limited to. Referring back to FIG. 1A, the homogenously mixed analyte capturing compound 52 is uniformly distributed and fixed on the surface of the reaction membrane 40. Similarly, referring to FIG. 2, the homogenously mixed complex formed by the analyte capturing compound 52 and the analyte/enzyme labeled compound 56 is uniformly distributed and fixed on the surface of the reaction membrane 40.

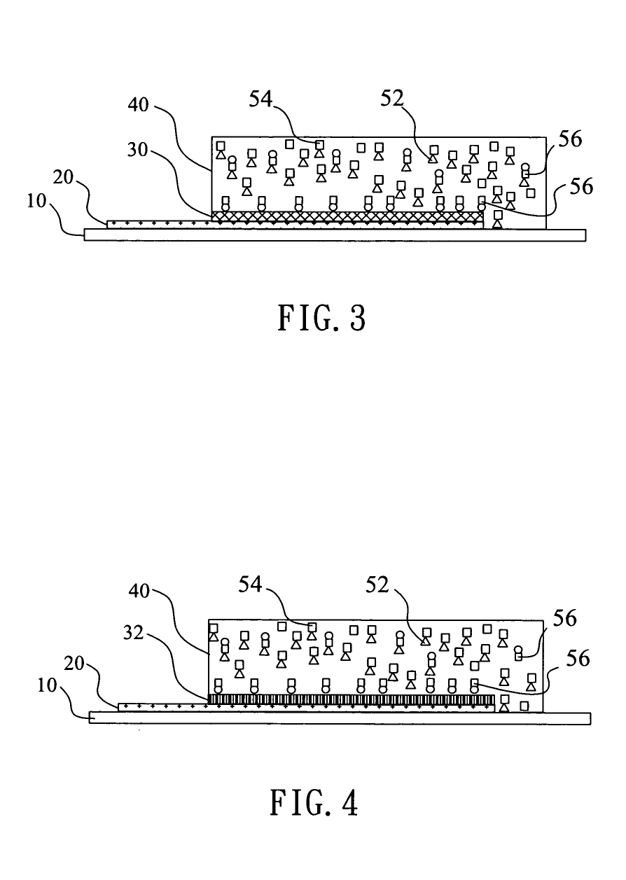

The reaction after the step of FIG. 1A is referring to FIG. 3, the specimen 55 is loaded onto the reaction membrane 40 through the specimen loading area 60 and flow toward the far end of the reaction membrane 40. On the other hand, the analyte/enzyme labeled compound 56 is re-dissolved by the specimen 55. Because of different affinity, the analyte 54 and the analyte/enzyme labeled compound 56 in the reaction membrane 40 will respectively react with the analyte capturing compound 52 to perform a competitive immunoreaction. In the case of small molecule of the analyte 54, the analyte/enzyme labeled compound 56, which is a conjugate of the analyte 54 and the catalyst 58, has a molecule weight and structure much larger than the analyte 54 does. Thus, the analyte capturing compound 52 prefers to combine with the analyte 54 first and then with the analyte/enzyme labeled compound 56. Accordingly, the analyte/enzyme labeled compound 56 is residual. Furthermore, as the amount of the analyte 54 of the specimen 55 is much higher, the analyte capturing compound 52 will almost totally conjugate with the analyte 54 and merely few bind with the analyte/enzyme labeled compound 56. Hence, there is more the analyte/enzyme labeled compound 56 residual. As the analyte is the huge molecule, the molecule weight of the analyte/enzyme labeled compound 56 is similar or a little larger than the analyte 54 so as the combination probability with the analyte capturing compound is depending on the quantity.

Following the above description and referring to FIG. 4, the residual and free analyte/enzyme labeled compound 56 will catalyze the electrochemical precursor 30 on the electrode area to produce the electrochemical active material 32. As the amount of the analyte 54 is higher, the more analyte/enzyme labeled compound 56 will be remained, the more electrochemical active material 32 is produced and the electrochemical signal is stronger as well. On the contrary, as the amount of the analyte 54 is low, the less analyte/enzyme labeled compound 56 is remained and the electrochemical active material 32 is produced few so as the electrochemical signal is weaker.

According to the description of FIG. 2 and referring to FIG. 3, the specimen 55 is the reaction membrane 40 through the specimen loading area 60 and flow toward the far end of the reaction membrane 40. On the other hand, the analyte/enzyme labeled compound 56 is re-dissolved by the specimen 55. In the case of the immuno substitution reaction for the analyte 54 of the small molecule, the analyte 54 replace the analyte/enzyme labeled compound 56 to bind with the analyte capturing compound 52. The analyte/enzyme labeled compound 56, which is a conjugate of the small molecule analyte 54 and the catalyst 58, has the molecule weight and structure much larger than the small molecule analyte 54. Therefore, the affinity of the analyte capturing compound 52 and the small molecule analyte 54 will higher than the affinity of the analyte capturing compound 52 and the analyte/enzyme labeled compound 56 so as the analyte/enzyme labeled compound 56 will be replaced by the analyte 54. The more the amount of the small molecule analyte 54 is, the more analyte/enzyme labeled compound 56 will be replaced.

Following the above description and referring to FIG. 4, the replaced analyte/enzyme labeled compound 56 will catalyze the electrochemical precursor 30 on the electrode area to produce the electrochemical active material 32. As the amount of the analyte 54 is higher, the more analyte/enzyme labeled compound 56 is replaced and the more electrochemical active material 32 is produced so as the electrochemical signal is stronger. On the contrary, as the amount of the analyte 54 is lower, the less analyte/enzyme labeled compound 56 is remained and the electrochemical active material 32 is replaced fewer so as the electrochemical signal is weaker.

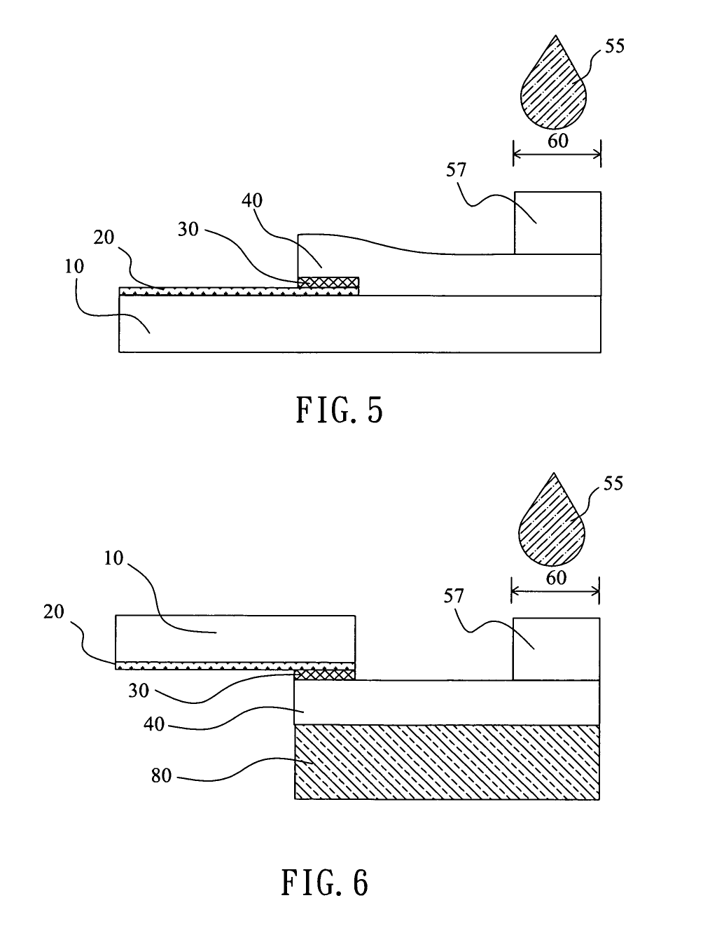

Referring to FIG. 5, the present preparation method includes the following steps. A screen-printed electrode 20 is formed on an insulating substrate 10, which is divided into an electrode area and a connecting area. An electrochemical precursor 30 is modified on the electrode area. A reaction membrane 40 has a second end stacked on the electrode area of the screen-printed electrode 20 and a first end configured for specimen loading area 60.

Besides, referring to FIG. 1A, the preparation method in one embodiment of the present further includes the following steps. An analyte capturing compound 52 is fixed in/on the reaction membrane 40. An analyte/enzyme labeled compound 56 is fixed in/on the specimen loading area 60. The specimen loading area 60 is configured for loading a specimen 55 having an analyte 54 therein. The analyte/enzyme labeled compound 56 is re-dissolved by the specimen 55 and moves toward the direction of the second end of the reaction membrane 40. A competitive immunoreaction is implemented by competing the analyte/enzyme labeled compound 56 with the analyte 54 in binding the analyte capturing compound 52. By capillary method, the residual analyte/enzyme labeled compound 56 is transported to the surface of the electrode area to catalyze the precursor 30 to produce the electrochemical active material 32, such as the shown in FIG. 4.

Further, referring to FIG. 2, in the case of small molecules of the analyte 54, preparation method in another embodiment of the present further includes the following steps. The complex of an analyte capturing compound 52 and an analyte/enzyme labeled compound 56 is fixed in/on the reaction membrane 40 with the side of the analyte capturing compound 52. The specimen loading area 60 is configured for loading a specimen 55 having an analyte 54 therein and then the specimen 55 proceeds toward the direction of the second end of the reaction membrane 40. The analyte 54 and the complex are performing a substitutive immunoreaction, wherein the residual and unbound analyte/enzyme labeled compound 56 is transported to the surface of the electrode area to catalyze the precursor 30 to produce the electrochemical active material 32, shown in FIG. 4.

It is a well-known technique for the electronic industry to manufacture the screen-printed electrode on the insulating substrate. For the preparation of the reaction membrane, the analyte capturing compound or the complex may be fixed on a surface of the reaction membrane by a dipping step in the reaction membrane. The present inventions may use the common sticking/adhering technology to stack the reaction membrane on the screen-printed electrode.

Referring to FIG. 5, the connecting area of the screen-printed electrode 20 of the electrochemical diagnostic sensing strip may be connected with a small instrument to measure the amount of the analyte by the electrochemical detection method, such as the cyclic voltammetry (CV), the linear stripping voltammetry (LSV), the square wave voltammetry (SWV) or the chronocoulometry (CC).

Referring to FIG. 6, the reaction membrane 40 is sticking with a supporting board 80 to enhance its support force of the structure. A second end of the reaction membrane 40 is stacking on the electrode area of the screen-printed electrode 20 and a specimen loading area 60 is arranged at a first end of the reaction membrane 40. For example, the actual thickness of the screen-printed electrode 20 and the electrochemical precursor 30 is much thinner than the insulating substrate 10 and the reaction membrane 40.

To sum up the forgoing, the electrochemical immuno diagnostic strip utilizes the immunoreaction to effectively overcome the interference problem and to enhance the detection sensitivity. Further, the present invention provides a single-step and prompt sensing strip and the quantitative analysis. Furthermore, the present invention combines the biotechnology and the electronic manufacturing technology to produce a biosensor strip, which is low-cost, simple and easy, and convenience.

While the present invention is susceptible to various modifications and alternative forms, a specific example thereof has been shown in the drawings and is herein described in detail. It should be understood, however, that the invention is not to be limited to the particular form disclosed, but on the contrary, the invention is to cover all modifications, equivalents, and alternatives falling within the spirit and scope of the appended claims.

What is claimed is:

1. An electrochemical immuno diagnostic strip comprising:
an insulating substrate;
a screen-printed electrode arranged on said insulating substrate, wherein said screen-printed electrode comprises an electrode area and a connecting area;
an electrochemical precursor modified on said electrode area; and
a reaction membrane, wherein a second end of said reaction membrane is stacked on said electrode area of said screen-printed electrode;
an analyte capturing compound fixed at said reaction membrane;
a specimen loading area at a first end of said reaction membrane for loading a specimen having an analyte therein, wherein an analyte/enzyme labeled compound is fixed on said specimen area and said analyte/enzyme labeled compound is re-dissolved by said specimen and proceeds toward the direction of said second end of said reaction membrane;
both of said analyte and said analyte/enzyme labeled compound performing an immunoreaction with said analyte capturing compound; and
said residual analyte/enzyme labeled compound transported to the surface of said electrode area for catalyzing said electrochemical precursor to produce a electrochemical active material.

2. The electrochemical immuno diagnostic strip according to claim 1, wherein said analyte is an antigen.

3. The electrochemical immuno diagnostic strip according to claim 2, wherein said analyte capturing compound is an antibody of said antigen.

4. The electrochemical immuno diagnostic strip according to claim 1, wherein said analyte is an antibody.

5. The electrochemical immuno diagnostic strip according to claim 4, wherein said analyte capturing compound is an antigen of said antibody.

6. The electrochemical immuno diagnostic strip according to claim 1, wherein said analyte/enzyme labeled compound is a conjugate of said analyte and a catalyst of said electrochemical precursor.

7. The electrochemical immuno diagnostic strip according to claim 1, wherein said electrochemical precursor is the para-aminophenyl β-D-galactopyranoside (PAPG).

8. The electrochemical immuno diagnostic strip according to claim 7, wherein said catalyst is the β-galactosidase.

9. An electrochemical immuno diagnostic strip comprising:
an insulating substrate;
a screen-printed electrode arranged on said insulating substrate, wherein said screen-printed electrode comprises an electrode area and a connecting area;
an electrochemical precursor modified on said electrode area;
a reaction membrane, wherein a second end of said reaction membrane is stacked on said electrode area on said screen-printed electrode;
a complex of an analyte capturing compound and an analyte/enzyme labeled compound fixed at said reaction membrane by the side of said analyte capturing compound of said complex;
a specimen loading area arranged at a first end of said reaction membrane for loading a specimen having an analyte therein, wherein said specimen proceeds toward the direction of said second end of said reaction membrane;
said analyte substitutes said analyte/enzyme labeled compound from complex; and
said replaced analyte/enzyme labeled compound transported toward the surface of said electrode area to catalyze said electrochemical precursor producing an electrochemical active material.

10. The electrochemical immuno diagnostic strip according to claim 9, wherein said analyte is an antigen.

11. The electrochemical immuno diagnostic strip according to claim 10, wherein said analyte capturing compound is an antibody of said antigen.

12. The electrochemical immuno diagnostic strip according to claim 9, wherein said analyte is an antibody.

13. The electrochemical immuno diagnostic strip according to claim 12, wherein said analyte capturing compound is an antigen of said antibody.

14. The electrochemical immuno diagnostic strip according to claim 9, wherein said analyte/enzyme labeled compound is a conjugate of said analyte and a catalyst of said electrochemical precursor.

15. The electrochemical immuno diagnostic strip according to claim 9, wherein said electrochemical precursor is the para-aminophenyl β-D-galactopyranoside (PAPG).

16. The electrochemical immuno diagnostic strip according to claim 15, wherein said catalyst is the β-galactosidase.

17. A preparation method of an electrochemical immuno diagnostic strip comprising the following steps:
providing an insulating substrate;
forming a screen-printed electrode on said insulating substrate, wherein said screen-printed electrode comprises an electrode area and a connecting area;
modifying an electrochemical precursor on said electrode area; and
providing a reaction membrane, wherein a second end of said reaction membrane is stacked on said electrode area on said screen-printed electrode and a specimen loading area is arranged at a first end of said reaction membrane.

18. The preparation method of an electrochemical immuno diagnostic strip according to claim 17, further comprising the following steps:
providing an analyte capturing compound fixed at said reaction membrane; and
providing an analyte/enzyme labeled compound fixed at said specimen loading area, wherein said specimen loading area is for applying a specimen having an analyte therein and said analyte/enzyme labeled compound is re-dissolved by said specimen and proceeds toward the direction of said second end of said reaction membrane;
an immunoreaction performed with said analyte capturing compound and both of said analyte and said analyte/enzyme labeled compound; and
said residual analyte/enzyme labeled compound transported to the surface of said electrode area for catalyzing said electrochemical precursor to produce said electrochemical active material.

19. The preparation method of an electrochemical immuno diagnostic strip according to claim 17, further comprises the following steps:
providing a complex of an analyte capturing compound and an analyte/enzyme labeled compound fixed at said reaction membrane by the side of said analyte capturing compound of said complex, wherein said specimen loading area is for applying a specimen having an analyte therein and said specimen is proceeding to the direction of said second end of said reaction membrane;
performing an immunoreaction with said complex and said analyte; and
producing a electrochemical active material, wherein said residual analyte/enzyme labeled compound is transported to the surface of said electrode area for catalyzing said electrochemical precursor to produce said electrochemical active material.

20. The preparation method of an electrochemical immuno diagnostic strip according to claim 18, wherein said analyte is an antigen.

21. The preparation method of an electrochemical immuno diagnostic strip according to claim 20, wherein said analyte capturing compound is an antibody of said antigen.

22. The preparation method of an electrochemical immuno diagnostic strip according to claim 18, wherein said analyte is an antibody.

23. The preparation method of an electrochemical immuno diagnostic strip according to claim 22, wherein said analyte is an antigen of said antibody.

24. The preparation method of an electrochemical immuno diagnostic strip according to claim 18, wherein said analyte/enzyme labeled compound is a conjugate of said analyte and a catalyst of said electrochemical precursor.

25. The preparation method of an electrochemical immuno diagnostic strip according to claim 18, wherein said electrochemical precursor is para-aminophenyl β-D-galactopyranoside (PAPG).

26. The preparation method of an electrochemical immuno diagnostic strip according to claim 25, wherein said catalyst is β-galactosidase.

27. The preparation method of an electrochemical immuno diagnostic strip according to claim 19, wherein said analyte is an antigen.

28. The preparation method of an electrochemical immuno diagnostic strip according to claim 27, wherein said analyte capturing compound is an antibody of said antigen.

29. The preparation method of an electrochemical immuno diagnostic strip according to claim 19, wherein said analyte is an antibody.

30. The preparation method of an electrochemical immuno diagnostic strip according to claim 29, wherein said analyte capturing compound is an antigen of said antibody.

31. The preparation method of an electrochemical immuno diagnostic strip according to claim 19, wherein said analyte/enzyme labeled compound is a conjugate of said analyte and a catalyst of said electrochemical precursor.

32. The preparation method of an electrochemical immuno diagnostic strip according to claim 19, wherein said electrochemical precursor is para-aminophenyl β-D-galactopyranoside (PAPG).

33. The preparation method of an electrochemical immuno diagnostic strip according to claim 32, wherein said catalyst is β-galactosidase.
